(19) United States
(12) Patent Application Publication
(10) Pub. No.: US 2017/0196901 A1
(43) Pub. Date: Jul. 13, 2017

(54) COMBINATION DRUG THERAPIES FOR CANCER AND METHODS OF MAKING AND USING THEM

(71) Applicant: Vicus Therapeutics, LLC, Morristown, NJ (US)

(72) Inventors: Newell Bascomb, Hendersonville, NC (US); John Maki, Mendham, NJ (US); Fredric Young, Los Altos, CA (US)

(21) Appl. No.: 15/405,106

(22) Filed: Jan. 12, 2017

Related U.S. Application Data
(60) Provisional application No. 62/278,110, filed on Jan. 13, 2016.

(51) Int. Cl. A61K 31/7068; A61K 31/407; A61K 31/337; A61K 31/138

(52) U.S. Cl. CPC A61K 31/7068; A61K 31/138; A61K 31/407; A61K 31/337; A61K 47/48284

(57) ABSTRACT

In alternative embodiments, provided are therapeutic combinations, pharmaceutical compositions, formulations, kits and devices for treating, preventing or ameliorating a tumor or a cancer, and methods for treating, preventing or ameliorating a tumor or a cancer. In alternative embodiments, provided are therapeutic combinations, pharmaceutical compositions, formulations, kits and devices comprising: a beta adrenergic receptor antagonist (a “beta blocker”) such as propranolol; a non-steroidal anti-inflammatory drug (a NSAID) such as etodolac; a gemcitabine or GEMZAR™; and, a taxane, a paclitaxel, TAXOL™, ONXOL™, an albumin-bound paclitaxel (nab-paclitaxel) or ABRAXANE™, or any equivalent thereof. In alternative embodiments, the therapeutic combinations further comprise an anti-cancer or anti-tumor antibody, a cytokine, and/or a chemotherapeutic agent.

### RELATED APPLICATIONS

This application claims benefit of priority to U.S. Provisional Patent Application Ser. No. (U.S. Ser. No.) 62/278,110, filed Jan. 13, 2016. The aforementioned application is expressly incorporated herein by reference in its entirety and for all purposes.

### FIELD OF THE INVENTION

This invention relates generally to medicine, pharmaceutical formulations and medical devices. In alternative embodiments, provided are therapeutic combinations, pharmaceutical compositions, formulations, kits and devices for treating, preventing or ameliorating a tumor or a cancer, and methods for treating, preventing or ameliorating a tumor or a cancer. In alternative embodiments, provided are therapeutic combinations, pharmaceutical compositions, formulations, kits and devices comprising: a beta adrenergic receptor antagonist (a “beta blocker”) such as propranolol; a non-steroidal anti-inflammatory drug (a NSAID) such as etodolac; a gemcitabine or GEMZAR™; and, a taxane, a paclitaxel, TAXOL™, ONXOL™, an albumin-bound paclitaxel (nab-paclitaxel) or ABRAXANE™, or any equivalent thereof. In alternative embodiments, the therapeutic combinations further comprise an anti-cancer or anti-tumor antibody, a cytokine, and/or a chemotherapeutic agent.

### BACKGROUND

Chemotherapy is important in cancer treatment, but chemotherapy drugs act by damaging high proliferating cells, and damage to normal cells results in chemotherapy toxicities and side effects. Chemotoxicity can be seen most in actively dividing tissues such bone marrow, hair follicles and gastrointestinal mucosa. New approaches in cancer chemotherapeutics are needed to address these challenges.

Nab-paclitaxel is a form of the chemotherapy drug paclitaxel that is bound to the human protein albumin and contained in nanoparticles. Binding paclitaxel to albumin eliminates the need for solvents that keep paclitaxel soluble once injected into the body but that can also cause allergic reactions and side effects. Albumin also plays a key role in delivering nutrients to dividing cells. Tumor cells require an abundance of nutrients to survive, so binding paclitaxel to albumin helps deliver paclitaxel to tumor cells.

Paclitaxel protein bound particles, or nab-paclitaxel, plus gemcitabine is becoming a standard of care for patients with metastatic adenocarcinoma of the pancreas (mPCa). In a randomized phase III trial, patients with metastatic pancreatic cancer who were treated with a combination of the albumin-bound paclitaxel (nab-paclitaxel Abraxane®) and gemcitabine (Gemzar®) lived longer than patients who were treated with gemcitabine alone. Patients who received both drugs also lived longer without their disease getting worse (progression-free survival). In another study, the combination of paclitaxel and gemcitabine was used as salvage therapy in metastatic breast cancer, and was associated with manageable toxicity, a high response rate, and remarkably prolonged survival durations. In another study, a regimen of nab-paclitaxel (Abraxane®) plus gemcitabine was as efficacious as the standard regimen in first-line treatment (gemcitabine alone) for metastatic pancreatic cancer, but less toxic and far less expensive.

In the United States, there were nearly 50,000 new cases of pancreatic cancer in 2014, which were responsible for about 40,000 deaths, representing 6.8% of all cancer deaths. The overall survival rate is 6.7%, but drops to 2.3% if it has metastasized. While administration of albumin-bound paclitaxel (nab-paclitaxel Abraxane®) and gemcitabine (Gemzar®) has increased median overall survival (OS) from 6.7 to 8.5 months, reducing the risk of death to about 28%, clearly other therapies and treatment modalities are needed to improve survival in this patient population.

### SUMMARY

In alternative embodiments, provided are therapeutic combinations of therapeutic agents or drugs for an individual in need thereof comprising or consisting of:

(a)

(i) a beta adrenergic receptor antagonist (a “beta blocker”);

(ii) a non-steroidal anti-inflammatory drug (a NSAID);

(iii) a gemcitabine or GEMZAR™; and,

(iv) a taxane, a paclitaxel, TAXOL™, ONXOL™, an albumin-bound paclitaxel (nab-paclitaxel) or ABRAXANE™, or any equivalent thereof;

(b) the therapeutic combination of therapeutic agents or drugs of (a), wherein the non-steroidal anti-inflammatory drug (a NSAID) comprises:
(i) a cyclooxygenase (COX) (or a prostaglandin synthase) inhibitor; or,
(ii) the COX inhibitor of (a), wherein the COX inhibitor comprises or consists of an etodolac or equivalent; a naproxen or equivalent; a celecoxib or equivalent; a rofecoxib or equivalent; a etoricoxib or equivalent; a valdecoxib or equivalent; a parecoxib or equivalent; a nabumetone or equivalent; a diclofenac (2-(2,6-dichloranilino) phenylacetic acid) or equivalent; or, a lumiracoxib or equivalent;

(c) the therapeutic combination of therapeutic agents or drugs of (a), wherein the beta adrenergic receptor antagonist (a beta blocker) comprises propranolol or equivalent, and optionally the propranolol is INDERAL™, AVLOCARDYL™, DERALIN™, DOCITON™, INDERALICI™, INNOPRAN XL™, or SUMIAL™; or

(d) the therapeutic combination of therapeutic agents or drugs of (a), wherein the therapeutic combination of an NSAID and beta blocker comprises or is a VT-122™ (Vicus Therapeutics, Morristown, N.J.).

In alternative embodiments, the etodolac is a LODINE™, LODINE SR™ or ECCOXOLAC™; or the celecoxib is CELEBREX™ or CELEBRA™; or the rofecoxib is VIOXX™, CEOXX™ or CEEOXX™; or the etoricoxib is ARCOXIA™, ALGIX™ or TAUXIB™; or the valdecoxib is BEXTRA™; the parecoxib is DYNASTAT™; the naproxen is XENOBID™, ALEVE™, ANAPROX™, MIRANAX™, NAPROGESIC™ NAPROSYN™, NAPRELAN™, PROXEN™ or SYNFLEX™; the nabumetone is RELAFEN™, RELIFEX™ or a GAMBARAN™; or, the diclofenac is FLECTOR PATCH™, VOLTAREN™, VOLTAROL™, DICLON™, DICLOFLEX DIFEN™ DIFENE™, CATAFLAM™, PENNSAID™, PANAMOR™, RHUMALGAN™, MODIFENAC™, ABITREN™, OLFEN™, VOVERAN™, ARTHROTEC™, DEDOLOR™, DEFLAMAT™, VETAGESIC™ or ZOLTEROL™.

In alternative embodiments, the therapeutic combination of therapeutic agents or drugs as provided herein further comprise an anti-cancer or anti-tumor antibody, wherein optionally the anti-cancer or anti-tumor antibody is an alemtuzumab, a brentuximab vedotin, a cetuximab, a gemtuzumab ozogamicin, an abritumomab tiuxetan, a nimotuzumab, an ofatumumab, a panitumumab, a rituximab, a tositumomab, or a trastuzumab.

In alternative embodiments, the therapeutic combination of therapeutic agents or drugs as provided herein further comprise a cytokine,

wherein optionally the cytokine comprises an IL-2 or an interferon (IFN),

and optionally the interferon is an alpha-IFN or a gamma-IFN;

and optionally the IL-2 is a recombinant IL-2, an aldesleukin, or a PROLEUKIN (Prometheus Laboratories),

wherein optionally the IL-2, recombinant IL-2, or aldesleukin is dosages at about: 1 to 20, 2 to 10, 4 to 5, or 4.5 millions of IUs per cycle; or is dosaged for: 1 to 5, 2 to 4, or 3 cycles number of cycles of therapy.

In alternative embodiments, the therapeutic combination of therapeutic agents or drugs as provided herein further comprise a chemotherapeutic agent,

wherein optionally the chemotherapeutic agent comprises a doxorubicin or a carboplatin, or comprises an inducer of apoptosis or a mitotic inhibitor or anti-microtubule inhibitor, or an alkylating agent, or a topoisomerase inhibitor, or a glycopeptide antibiotic, or steroid receptor inhibitor, or a matrix metalloproteinase (MMP) inhibitor, or an mTOR (mammalian target of rapamycin) inhibitor, or a macrolide or a composition comprising a macrolide ring,

and optionally the inducer of apoptosis or a mitotic inhibitor or anti-microtubule inhibitor comprises or consists of a raltitrexed or equivalent, or TOMUDEX™; a doxorubicin or equivalent, or ADRIAMYCIN™; a fluorouracil or 5-fluorouracil or equivalent; a docetaxel or equivalent, or TAXOTERE™; a larotaxel, tesetaxel or ortataxel or equivalent; an epothilone or an epothilone A, B, C, D, E or F or equivalent; an ixabepilone (also known as azaepothilone B) or equivalent, or BMS-247550™; a vincristine (also known as leurocristine) or equivalent, or ONCOVIN™; a vinblastin, vinblastine, vindesine, vinflunine, vinorelbine or NAVELBINE™ or equivalent; or, any combination thereof,

and optionally the alkylating agent comprises or consists of a cisplatin or equivalent; a cisplatinum or equivalent; a cis-diamminedichloridoplatinum(II) (CDDP) or equivalent; a carboplatin or equivalent; a oxaloplatin or equivalent; a cyclophosphamide (cytophosphane) or equivalent, or ENDOXAN™, CYTOXAN™, NEOSAR™ or REVIMMUNE™; a mechlorethamine or equivalent; a chlormethine or equivalent; a mustine or equivalent; a nitrogen mustard or equivalent; a chlorambucil or equivalent, or LEUKERAN™; or, a combination thereof,

and optionally the topoisomerase inhibitor comprises or consists of an etoposide or equivalent, or EPOSIN™, ETOPOPHOS™, VEPESID™ or VP-16™; an amsacrine or equivalent; a topotecan or equivalent, or HYCAMTIN™; a teniposide or equivalent, or VUMON™ or VM-26™; an epipodophyllotoxin or equivalent; a camptothecin or equivalent; an irinotecan or equivalent, or CAMPTOSAR™; or, a combination thereof, and optionally the glycopeptide antibiotic comprises or consists of a bleomycin or equivalent or a bleomycin A2 or B2 or equivalent; a mitomycin or a mitomycin C or equivalent, a plicamycin (also known as mithramycin) or equivalent, or MITHRACIN™; or, a combination thereof,

and optionally the steroid receptor inhibitor comprises or consists of an estrogen receptor modulator (a SERM), and optionally the estrogen receptor modulator comprises or consists of a tamoxifen or equivalent, or NOLVADEX™, ISTUBAL™ or VALODEX™, and optionally the steroid inhibitor or an anti-steroid comprises or consists of a finasteride or equivalent, or PROSCAR™, PROPECIA™, FINCAR™, FINPECIA™, FINAX™, FINAST™, FINARA™, FINALO™, PROSTERIDE™, GEFINA™, APPECIA™ FINASTERID IVAX™, FINASTERID or ALTERNOVA™,

and optionally the macrolide or composition comprising a macrolide ring comprises or consists of a clarithromycin or equivalent, or BIAXIN™, KLARICID™, KLABAX™, CLARIPEN™, CLARIDAR™, FROMILID™ or CLACID™; an azithromycin or equivalent, or ZITHROMAX™, ZITROMAX™ or SUMAMED™; a dirithromycin or equivalent; an erythromycin or equivalent; a roxithromycin or equivalent, or ROXO™, SURLID™, RULIDE™, BIAXSIG™, ROXAR™, ROXIMYCIN™ or COROXIN™; a telithromycin or equivalent or KETEK™; a josamycin or equivalent; a kitasamycin or equivalent; a midecamycin or equivalent; oleandomycin or equivalent; a roxithromycin or equivalent, or ROXO™, SURLID™, RULIDE™, BIAXSIG™, ROXAR™, ROXIMYCINTM or COROXIN™; a troleandomycin or equivalent; or a tylosin or equivalent; or, any combination thereof,

wherein optionally the chemotherapeutic agent comprises a sorafenib or equivalent, or NEXAVAR™; a sunitinib or equivalent, or SUTENT™; an erlotinib or equivalent, or TARCEVA™; an imatinib or equivalent, or GLEEVEC™; a lapatinib or equivalent, or TYKERB™; a toceranib or equivalent, or PALLADIA™; a masitinib or equivalent, or MASIVET™, a bevacizumab or equivalent, or AVASTIN™; a trastuzumab or equivalent, or HERCEPTIN™; a cetuximab or equivalent, or ERBITUX™; a bevacizumab or equivalent, or AVASTIN™ or BIBW 2992; a gefitinib or equivalent, or IRESSA™; a ranibizumab or equivalent, or LUCENTIS™; a pegaptanib or equivalent, or MACUGEN™; a dasatinib or equivalent, or BMS-354825™; a sunitinib or equivalent, or SUTENT™; a pazopanib or equivalent; a nilotinib or equivalent, or TASIGNA™; a panitumumab or equivalent, or VECTIBIX™; a bandetinib or equivalent; a brivanib or equivalent, or E7080™; a thalidomide or equivalent, or THALOMID™; lenalidomide or equivalent, or REVLIMID™; a bortezomib or equivalent, or VELCADE™; disulfiram or equivalent, or ANTABUSE™ or ANTABUS™; or an epigallocatechin gallate (EGCG) or equivalent; a demecolcine, an etoglucid or elsamitrucin, a lonidamine, a lucanthone, a mitotane or a mitoguazone or equivalent; or any combination thereof.

In alternative embodiments, the therapeutic combination of therapeutic agents or drugs as provided herein further comprise a radiotherapy enhancing agent. In alternative embodiments, the therapeutic combination of therapeutic agents or drugs as provided herein further comprise a proton pump inhibitor (a PPI), wherein optionally the proton pump inhibitor comprises or consists of a benzimidazole compound or structure, or an imidazopyridine compound or structure.

In alternative embodiments, the therapeutic combination of therapeutic agents or drugs as provided herein further comprise a radioactive particle or isotope; or a microscopic, radioactive glass microsphere, or a TheraSphere (THERASPHERE™); or a drug-eluting or a cancer drug-eluting particle, liposome or bead, or a doxorubicin-loaded drug-eluting bead, or a DC Bead®.

In alternative embodiments, the therapeutic combination of therapeutic agents or drugs as provided herein further comprise an adjuvant.

In alternative embodiments, two or more drugs of the therapeutic combination are formulated as separate compositions, or two or more drugs of the therapeutic combination are formulated into one composition or drug formulation (two or more drugs of the therapeutic combination are formulated together).

In alternative embodiments, the beta adrenergic receptor antagonist, or the beta blocker or equivalent, or the propranolol or equivalent; the non-steroidal anti-inflammatory drug, or the NSAID or equivalent, or the etodolac or equivalent; the gemcitabine or GEMZAR™; and the taxane, the paclitaxel, TAXOL™, ONXOL™, the albumin-bound paclitaxel (nab-paclitaxel) or ABRAXANE™, or any equivalent thereof, are all formulated or packaged in different compositions or formulations, in a single formulation or package, or in paired combinations or packages,

and optionally are all administered separately or are all administered together,

and optionally the beta adrenergic receptor antagonist, the beta blocker or equivalent, or the propranolol or equivalent and the NSAID or equivalent, or the etodolac or equivalent, are administered or packaged together either in separate formulations or packages or a single formulation or package,

and optionally the gemcitabine or GEMZAR™; and the taxane, the paclitaxel, TAXOL™, ONXOL™, the albumin-bound paclitaxel (nab-paclitaxel) or ABRAXANE™ or any equivalent thereof, are administered or packaged together either in separate formulations or packages or a single formulation or packages;

(b) the beta adrenergic receptor antagonist, or a beta blocker or equivalent, or a propranolol or equivalent; and the non-steroidal anti-inflammatory drug, or a NSAID or equivalent, or an etodolac or equivalent are formulated in different compositions or formulations, or, are formulated in the same composition or formulation, or are formulated together.

In alternative embodiments, one or two or more or all of the drugs of the therapeutic combination are packaged individually, or are packaged together, or packaged in any combination, in a single package, a plurality of packages or packettes, or packaged as a blister packet, lidded blister or blister card or packets, or a shrink wrap.

In alternative embodiments, the beta adrenergic receptor antagonist, or a beta blocker or equivalent, or a propranolol or equivalent; the non-steroidal anti-inflammatory drug, or a NSAID or equivalent, or an etodolac or equivalent; and the therapeutic agent for the treatment of cancer, are packaged individually in a single package, a plurality of packages or packettes, or a blister packet, lidded blister or blister card or packets, or a shrink wrap. In alternative embodiments, one or two or more or all of the drugs of the therapeutic combination are packaged together or in any combination in a single package, a plurality of packages or packettes, or a blister packet, lidded blister or blister card or packets, or a shrink wrap. In alternative embodiments, two, three or more or all of the drugs are released upon opening of the single package, plurality of packages or packettes, blister packet, lidded blister, blister card or packets or shrink wrap.

In alternative embodiments, the beta adrenergic receptor antagonist, or a beta blocker or equivalent, or a propranolol or equivalent; the non-steroidal anti-inflammatory drug, or a NSAID or equivalent, or an etodolac or equivalent; and the therapeutic agent for the treatment of cancer are packaged together in a single package, a plurality of packages or packettes, or a blister packet, lidded blister or blister card or packets, or a shrink wrap, and two or more or all of the drugs are released upon opening of the single package, plurality of packages or packettes, blister packet, lidded blister, blister card or packets or shrink wrap.

In alternative embodiments, one or two or more or all of the drugs of the therapeutic combination are formulated or manufactured as a parenteral formulation, an aqueous solution, a liposome, an injectable solution, a tablet, a pill, a lozenge, a capsule, a caplet, a patch, a spray, an inhalant, a powder, a freeze-dried powder, an inhalant, a patch, a gel, a geltab, a nanosuspension, a nanoparticle, a nanoliposome, a microgel, a pellet, a suppository or any combination thereof.

In alternative embodiments, one or two or more or all of the drugs of the therapeutic combination are formulated or manufactured together in one parenteral formulation, one aqueous solution, one liposome, one injectable solution, one freeze-dried powder, one feed, one food, one food supplement, one pellet, one lozenge, one liquid, one elixir, one aerosol, one inhalant, one adhesive, one spray, one powder, one freeze-dried powder, one patch, one tablet, one pill, one capsule, one gel, one geltab, one lozenge, one caplet, one nanosuspension, one nanoparticle, one nanoliposome, one microgel or one suppository.

In alternative embodiments: (a) the dosage of etodolac ranges from about 200 mg to 400 mg a day, or, about 10, 15, 20, 25, 30, 35, 40, 45, 50, 75, 80, 85, 90, 100, 150, 200, 250, 300, 350, 400, 450, 500, 600, 700, 800, 900 or 1000 mg or more; or, (b) the dosage of propranolol ranges from 10 to 320 mg per day based on heart rate and blood pressure of the individual, or, about 10, 15, 20, 25, 30, 35, 40, 45, 50, 75, 80, 85, 90, 100, 150, 200, 250, 300, 350, 400, 450, 500, 600, 700, 800, 900 or 1000 mg or more.

In alternative embodiments, the drug combination is packaged in dosages that match a chrono-dosing regimen to match an optimal dose for the time of day. In alternative embodiments, the beta adrenergic receptor antagonist or a beta blocker or equivalent, or a propranolol or equivalent; the non-steroidal anti-inflammatory drug or NSAID or equivalent, or etodolac or equivalent; and also optionally the gemcitabine or GEMZAR™ and/or the taxane, the paclitaxel, TAXOL™, ONXOL™, the albumin-bound paclitaxel (nab-paclitaxel) or ABRAXANE™, or any equivalent thereof, are packaged in dosages that match a chrono-dosing regimen to match an optimal dose for the time of day.

In alternative embodiments, the beta adrenergic receptor antagonist or beta blocker or equivalent or propranolol or equivalent; the non-steroidal anti-inflammatory drug or NSAID or equivalent or etodolac or equivalent; and also optionally the gemcitabine or GEMZAR™ and/or the taxane, the paclitaxel, TAXOL™, ONXOL™, the albumin-bound paclitaxel (nab-paclitaxel) or ABRAXANE™, or any equivalent thereof, are packaged in dosages that match a chrono-dosing regimen comprising:

(a) in the AM, 20 mg beta adrenergic receptor antagonist (a beta blocker), e.g., a propranolol or equivalent, 200mg NSAID, e.g., an etodolac or equivalent; in the afternoon, 10 mg beta blocker, 200 mg NSAID, e.g., an etodolac or equivalent; in the PM, 10 mg beta blocker, 400 mg NSAID;

(b) in the AM 40 mg beta adrenergic receptor antagonist (a beta blocker), e.g., a propranolol or equivalent, 200 mg NSAID, e.g., an etodolac or equivalent; in the afternoon 20 mg beta blocker, 200 mg NSAID; in the evening, 20 mg propranolol, 400 mg NSAID;

(c) in the AM 80 mg beta adrenergic receptor antagonist (a beta blocker), e.g., a propranolol or equivalent, 200 mg NSAID; in the afternoon 40 mg beta blocker, 200 mg NSAID, in the evening 40 mg, NSAID; or

(d) a dose escalation comprising a regimen of (a) to (b) to (c).

In alternative embodiments, the beta adrenergic receptor antagonist or beta blocker or equivalent or propranolol or equivalent; the non-steroidal anti-inflammatory drug or NSAID or equivalent or etodolac or equivalent; and also optionally the gemcitabine or GEMZAR™ and/or the taxane, the paclitaxel, TAXOL™, ONXOL™, the albumin-bound paclitaxel (nab-paclitaxel) or ABRAXANE™, or any equivalent thereof, are packaged in dosages that match a chrono-dosing regimen comprising:

Start: AM, 20 mg propranolol, 200 mg etodolac; afternoon, 10 mg propranolol, 200 mg etodolac; PM 5 mg propranolol, 400 mg etodolac;

Dose Escalation 1: AM 40 mg propranolol, 200 mg etodolac; afternoon 20 mg propranolol, 200 mg etodolac; evening, 10 mg propranolol, 400 mg etodolac;

Dose escalation 2: AM 80 mg propranolol, 200 mg etodolac; afternoon 40 mg propranolol, 200 mg etodolac, evening 20 mg, etodolac.

In alternative embodiments, the therapeutic drug combination is formulated for administration once a day, b.i.d. or t.i.d., or weekly, or biweekly, or monthly. In alternative embodiments, the beta adrenergic receptor antagonist (a beta blocker) or propranolol or equivalent; the non-steroidal anti-inflammatory drug or NSAID or etodolac or equivalent; and also optionally the gemcitabine or GEMZAR™ and/or the taxane, the paclitaxel, TAXOL™, ONXOL™, the albumin-bound paclitaxel (nab-paclitaxel) or ABRAXANE™ or any equivalent thereof, are formulated for administration once a day, b.i.d. or t.i.d, or weekly, or biweekly, or monthly.

In alternative embodiments, the therapeutic combination of drugs are formulated for administration intravenously, topically, orally, by inhalation, by infusion, by injection, by inhalation, intraperitoneally, intramuscularly, subcutaneously, intra-aurally, for intra-articular administration, for intra-mammary administration, for topical administration or for absorption through epithelial or mucocutaneous linings.

In alternative embodiments, provided are: a device, a medical device, an implant, a breast implant, a prosthesis, a stent, a catheter, comprising a therapeutic combination of therapeutic agents or drugs as provided herein.

In alternative embodiments, provided are: a pharmaceutical composition or formulation comprising the therapeutic combination as provided herein; and the pharmaceutical composition or formulation can further comprise a pharmaceutically acceptable excipient. In alternative embodiments, the pharmaceutical composition or formulation is formulated or manufactured as a feed, a food, a food or feed concentrate, a pellet, a lozenge, a liquid, a lotion, an implant, a nanoparticle, an elixir, an aerosol, a spray, an aerosol, an inhalant, a powder, a tablet, a pill, a capsule, a gel, a geltab, a nanosuspension, a nanoparticle, a patch, a microgel or a suppository.

In alternative embodiments, provided are method and uses for treating, preventing or ameliorating a tumor or a cancer, comprising: applying or administering to an individual in need thereof; or, applying or administering to an effected tissue: the therapeutic combination as provided herein, or a pharmaceutical composition or formulation as provided herein, wherein optionally the therapeutic agents or drugs are administered separately or together, or at the same time, or in synchrony, or by chrono-dosing, or one of the therapeutic agents or drugs is administered before another of the therapeutic agents or drugs, and optionally the therapeutic agents or drugs are formulated for administration intravenously (IV), parenterally, nasally, topically or locally, orally, or by liposome, implant or vessel-targeted nanoparticle delivery.

In alternative embodiments of the methods and uses, the cancer or tumor is: a pancreatic cancer, an adenocarcinoma, a mastocytoma or a mast cell tumor, an ovarian cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm's tumor, ovarian carcinoma, a bone cancer, an osteosarcoma, a renal cancer, or head and neck cancer, oral cancer, a laryngeal cancer, or an oropharyngeal cancer.

In alternative embodiments, provided are methods for treating, preventing or ameliorating a tumor or a cancer, comprising:

(a) applying or administering to an individual in need thereof; or, applying or administering to an effected tissue; the therapeutic combination as provided herein, or a pharmaceutical composition or formulation as provided herein,

wherein optionally the therapeutic agents or drugs are administered separately or together, or at the same time, or in synchrony, or by chrono-dosing, or one of the therapeutic agents or drugs is administered before another of the therapeutic agents or drugs,

and optionally the therapeutic agents or drugs are formulated for administration intravenously (IV), parenterally, nasally, topically or locally, orally, or by liposome, implant or vessel-targeted nanoparticle delivery; and

(b) administering to the individual in need thereof:
(i) a systemic anti-cancer or anti-tumor treatment, wherein optionally the systemic anti-cancer or anti-tumor treatment comprises administration of a drug, a biologic, a nutrient, an anti-cancer or anti-tumor dietary regimen, a radioactive agent, a tumor ablative agent, or
(ii) an anti-cancer or anti-tumor radiotherapy or a proton beam therapy,

wherein the therapeutic combination or pharmaceutical composition or formulation of (a) is administered before the anti-cancer or anti-tumor treatment of (b), or both are administered consecutively, or the therapeutic combination or pharmaceutical composition or formulation of (a) is administered after the anti-cancer or anti-tumor treatment of (b), or any combination thereof.

In alternative embodiments, the method and uses can further comprise (or comprise use of): an anti-cancer or anti-tumor radiotherapy or a proton beam therapy.

In alternative embodiments, provided are Uses of the therapeutic combination as provided herein in the manufacture of a medicament. In alternative embodiments, provided are Uses of the therapeutic combination as provided herein in the manufacture of a medicament for treating a cancer or a tumor. In alternative embodiments of the uses and methods, the cancer or tumor is: a pancreatic cancer, an adenocarcinoma, a mastocytoma or a mast cell tumor, an ovarian cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a Histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm' s tumor, ovarian carcinoma, a bone cancer, an osteosarcoma, a renal cancer, or head and neck cancer, oral cancer, a laryngeal cancer, or an oropharyngeal cancer.

The details of one or more aspects of the invention are set forth in the description below. Other features, objects, and advantages of the invention will be apparent from the description and from the claims.

All publications, patents and patent applications cited herein are hereby expressly incorporated by reference for all purposes.

### DESCRIPTION OF DRAWINGS

The drawings set forth herein are illustrative of embodiments described herein and are not meant to limit the scope of the invention as encompassed by the claims.

FIG. 1 schematically illustrates the potential effects of adrenergic signal inhibition by propranolol on inflammation and immune function, where the inhibited pathways are noted with an “X”.

FIG. 2 schematically illustrates the potential effects of COX-2 inhibition by etodolac on inflammation and immune function, where the inhibited pathways are noted with an “X”.

FIG. 3 schematically illustrates the study design of a randomized, open label study, where patients were randomized to receive either: (1) a placebo in addition to the gemcitabine and nab-paclitaxel (“GemNab”) combination (“the GemNab arm”); or, (2) administered the exemplary therapeutic combination of: gemcitabine and nab-paclitaxel (“GemNab”) and chrono-dosed propranolol and etodolac (PE) (“PEGenNab”) after one week of propranolol and etodolac (PE) alone (“the PEGemNab arm”), as described in detail in Example 1, below.

FIG. 4 graphically illustrates a Kaplan-Meier estimate of overall survival of patients in the clinical study described in detail in Example 1, below, where overall survival in the VT-122-treated arm (2) was 17 months, which was significantly greater than the 9.3 months of the control arm (1).

FIG. 5 graphically illustrates data from the clinical study described in detail in Example 1, below, including the maximum weight gain of patients in the study, which was significantly greater in the VT-122 chronodosed group (arm 2).

FIG. 6 graphically illustrates data from the clinical study described in detail in Example 1, below, where patients in the VT-122 chronodosed group (arm 2) had significantly less grade 2 or 3 neuropathy; and time to a 20% increase in visual analog pain scale score was significantly longer in the VT-122 (arm 2) group.

Like reference symbols in the various drawings indicate like elements.

### DETAILED DESCRIPTION

In alternative embodiments, provided are therapeutic combinations, pharmaceutical compositions, formulations, kits and devices for treating, preventing or ameliorating a tumor or a cancer, and methods for treating, preventing or ameliorating a tumor or a cancer. In alternative embodiments, provided are therapeutic combinations, pharmaceutical compositions, formulations, kits and devices comprising: a beta adrenergic receptor antagonist (a “beta blocker”); a non-steroidal anti-inflammatory drug (a NSAID); a gemcitabine or GEMZAR™; and, a taxane, a paclitaxel, TAXOL™, ONXOL™, an albumin-bound paclitaxel (nab-paclitaxel) or ABRAXANE™, or any equivalent thereof In alternative embodiments, the therapeutic combinations further comprise an anti-cancer or anti-tumor antibody, a cytokine, and/or a chemotherapeutic agent.

In alternative embodiments, the tumor or cancer treated is a pancreatic cancer, an adenocarcinoma, a metastatic adenocarcinoma of the pancreas (mPCa). Although for pancreatic cancer treatments administration of albumin-bound paclitaxel (nab-paclitaxel Abraxane®) and gemcitabine (Gemzar®) has increased median overall survival (OS) from 6.7 to 8.5 months and reduced the risk of death to about 28%, clearly other therapies and treatment modalities are needed to improve survival in this patient population, and exemplary therapeutic combinations of this invention do address this issue and solve this problem, as demonstrated by a study described in Example 1, below.

In alternative embodiments the cancer is a dysfunctional cell condition. In alternative embodiments the cancer or dysfunctional cell condition comprises (is) any metastatic or benign tumor, and the methods or uses as provided herein are used for ameliorating, treating (killing, eliminating, stopping the growth and/or metastasis of) cancer stem cells or cancer cells from: a pancreatic cancer, an adenocarcinoma, a metastatic adenocarcinoma of the pancreas (mPCa), lung cancer, bone cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, a neoplasm of the central nervous system (CNS), primary CNS lymphoma, spinal axis tumors, brain stem glioma or pituitary adenoma, and any combination thereof.

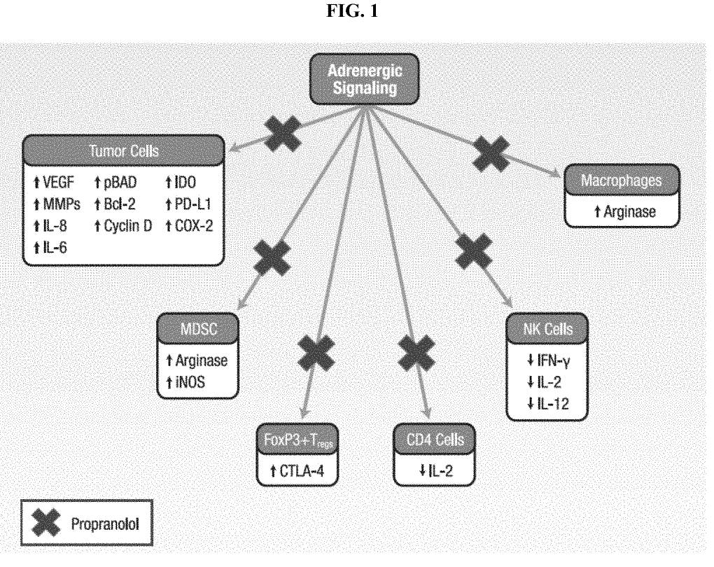

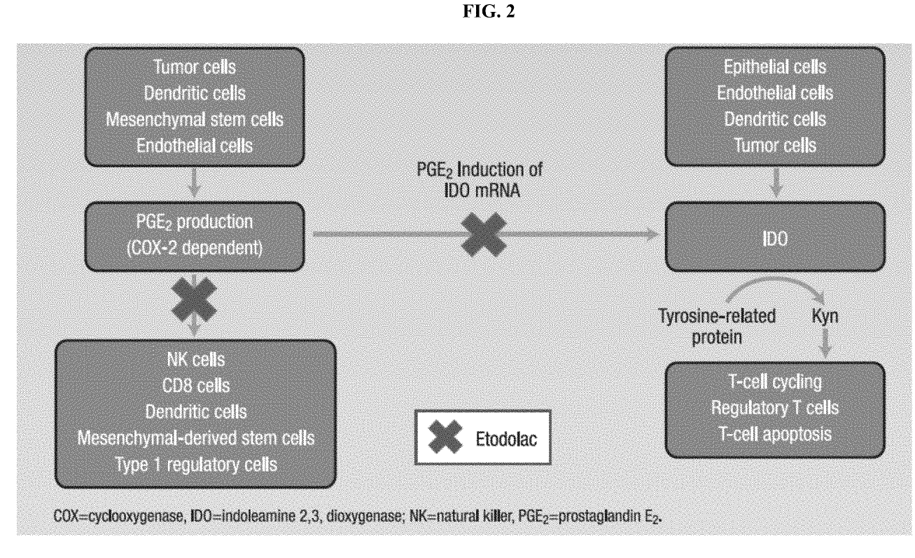

While the invention is not limited by any particular mechanism of action, preclinical studies have shown that propranolol, a beta blocker developed for cardiovascular conditions, and etodolac, a COX-2 selective nonsteroidal anti-inflammatory developed for pain management of osteoarthritis, effectively block adrenergic and prostaglandin signaling pathways, respectively, can dampen tumor promoting inflammation and switch immune states, e.g., as illustrated in FIG. 1 and FIG. 2; where FIG. 1 schematically illustrates the potential effects of adrenergic signal inhibition by propranolol on inflammation and immune function, where the inhibited pathways are noted with an “X”; and FIG. 2 schematically illustrates the potential effects of COX-2 inhibition by etodolac on inflammation and immune function, where the inhibited pathways are noted with an “X”. While the invention is not limited by any particular mechanism of action, VT-122 (Vicus Therapeutics, Morristown NJ), comprising both propranolol and etodolac, inhibits adrenergic beta receptors 1-2, COX-2 & TRPA1 and the MAPK, PI3K, PKA, STAT3 & nociception signaling cascades.

While the invention is not limited by any particular mechanism of action, by chrono-modulating multiple signaling cascades in the tumor microenvironment and the tumor-induced systemic environment, VT-122:
Damps tumor promoting inflammation: inhibits perineural invasion, metastasis, angiogenesis, lymphangiogenesis, fibrogenesis and gluconeogenesis.
Restores immune surveillance: reduces myeloid derived suppressor cells (MDSCs), activates NK cells and switches tumor-associated macrophages (TAMs) and T-cells from M2/Th2 to M1/Th1 state
Improves treatment tolerance: reduces inflammation potentiated hand-foot-syndrome, chemotherapy-induced-peripheral-neuropathy and cachexia.

While the invention is not limited by any particular mechanism of action, VT-122 is differentiated from other immunotherapies along several dimensions because administration of VT-122 can:
Target multiple un-drugged pathways within the tumor microenvironment and tumor-induced systemic environment.
Modulate both the innate and adaptive immune systems.
Reduce side-effects of sorafenib in liver cancer and GemNab in pancreatic cancer.
Enable patient adherence to the right drugs at the right doses and at the right times with a unique controlled release formulation and blister card packaging system.

In alternative embodiments provided are products of manufacture comprising a blister package, a lidded blister or a blister card or packet, a clamshell, a tray or a shrink wrap comprising a therapeutic combination as provided herein, or the pharmaceutical composition or formulation as provided herein. In alternative embodiments the products of manufacture can comprise a blister package, a lidded blister or a blister card or packet, a clamshell, a tray or a shrink wrap comprising a therapeutic combination as provided herein, or the pharmaceutical composition or formulation as provided herein, wherein the therapeutic combination or pharmaceutical composition or formulation are manufactured and/or formulated for at least two, three, four or five or more dosage administrations; or the therapeutic combination or pharmaceutical composition or formulation are manufactured and/or formulated for once a day, or b.i.d. (twice a day), or t.i.d. (three times a day), or four times a day, administration.

In alternative embodiments, a drug combination as provided herein is formulated, packaged or designed for drug regimen compliance of a cancer patient population, a pediatric or geriatric population, or a mentally compromised patient population. In alternative embodiments drug combination(s) as provided herein are formulated, packaged or designed for drug regimen compliance of a cancer patient population having mild or severe mental retardation, slow cognition, dementia, senility, Alzheimer's disease, traumatic brain injury, chemical brain damage, mental diseases (e.g., dissociative disorder, obsessive-compulsive disorder, delusional disorder, schizophrenia, mania, panic disorder, depression, dyslexia, any learning disability and the like) post-traumatic stress disorder, traumatic war neurosis, post-traumatic stress syndrome (PTSS), physical disability (e.g., blindness).

In alternative embodiments of the products of manufacture as provided herein the therapeutic combination or pharmaceutical composition or formulation are formulated (e.g., manufactured) as one dosage administration in the morning and one dosage administration in the evening; or are formulated as one dosage administration in the morning, one dosage mid-day and one dosage administration in the evening. In one aspect, the dosage schedule provides a relatively higher dose of one drug in the morning (the AM) than in the evening, and a relatively higher dose of another medication in the evening than in the morning. For example, in alternative embodiments the therapeutic combination or the pharmaceutical composition are formulated for multiple administrations, e.g., at least two administrations, one in the morning and one in the evening, wherein the dosage schedule provides a relatively higher dose of beta blocker in the morning (the AM) than in the evening, and a relatively higher dose of an anti-inflammatory medication in the evening than in the morning.

In alternative embodiments, the products of manufacture or formulations as provided herein comprise a therapeutic combination as provided herein or the pharmaceutical composition or formulation as provided herein, and a nutritional supplement, or food supplement or feed supplement.

### Methods of Administration

In alternative embodiments, provided herein are therapeutic combinations of drugs, pharmaceutical compositions, preparations and kits, that can be administered by several routes, for formulated for administration by any of several routes, including intravenous, topical and oral, subcutaneous, mucosal, aerosol, or combinations thereof. In exemplary alternative embodiments, the therapeutic combination of drugs are formulated for administration intravenously, topically, orally, by inhalation, by infusion, by injection, by inhalation, intraperitoneally, intramuscularly, subcutaneously, intra-aurally, for intra-articular administration, for intra-mammary administration, for topical administration or for absorption through epithelial or mucocutaneous linings.

The invention provides a device, a medical device, an implant, a breast implant, a prosthesis, a stent, a catheter, comprising a therapeutic combination of therapeutic agents or drugs of the invention.

For example, one embodiment comprises a product of manufacture comprising a pharmaceutical composition or a formulation, a blister package, a lidded blister or a blister card or packet, a clamshell, a tray or a shrink wrap, or a kit, comprising: therapeutic combinations of drugs, pharmaceutical compositions or preparations as provided herein.

In alternative embodiments, although all ingredients can be in one blister package, a lidded blister or a blister card or packet, a clamshell, a tray or a shrink wrap, or a kit, separate ingredients can be formulated e.g., for topical application, for oral or for topical application. Each ingredient can be either separately packaged, or can be formulated as one unit dose, e.g., as one tube (e.g., with gel, lotion etc.), ampoule, blister packette and the like.

In alternative embodiments, provided herein are forms of compositions, preparations and kits that can be administered by inhalation, infusion or injection, (e.g., intraperitoneal, intramuscular, subcutaneous, intra-aural, intra-articular, intra-mammary, etc.), topical application (e.g., on areas, such as eyes, ears, skin or on afflictions such as wounds, burns, etc.), and by absorption through epithelial or mucocutaneous linings (e.g. vaginal and other epithelial linings, gastrointestinal mucosa, etc.). Methods are known for making compositions, preparations and kits containing the present components that are suitable for each of these methods of administration as well as other methods of administration that are known in the art.

In alternative embodiments, provided herein are compositions, preparations and kits in liquid forms that can be administered orally. The compositions, preparations and kits can be also prepared as capsules, gels, geltabs, tablets, powders, sprays, aerosols, pellets (e.g. for animal consumption), suppositories, lotions, patches or adhesives (e.g., for the skin), or creams and ointments. The compositions, preparations and kits can be also prepared as physiological solutions suitable for I.V. administration or other parenteral administration.

In one aspect, a multi-ingredient kit as provided herein comprises (contains) two or more ingredients. An amount may be determined, e.g. by mass or by weight or by molar amount. In another aspect, a multi-ingredient kit may contain two or more ingredients in unequal amounts. In another aspect, a multi-ingredient kit may contain two or more ingredients in approximately equal amounts and/or one or more ingredients that are not in unequal amounts.

In another embodiment, said multi-ingredient kit may contain two or more ingredients that are admixed. In another aspect, said multi-ingredient kit may contain two or more ingredients that are not admixed. In another aspect, said multi-ingredient kit may contain two or more ingredients that are partially admixed. In another aspect, said multi-ingredient kit may contain two or more ingredients that are at least partially admixed, as well as one or more ingredients that are not admixed. An ingredient in a multi-ingredient kit may be liquid forms that can be administered orally.

### Dosaging

In alternative embodiments of the therapeutic combination, the gemcitabine and nab-paclitaxel (“GemNab”) are administered using standard dosage and administration, i.e., dosaging and administration regimen as on the FDA approved label (the FDA has approved nab-paclitaxel (ABRAXANE™) plus gemcitabine as a first-line treatment for patients with metastatic adenocarcinoma of the pancreas).

In alternative embodiments of the therapeutic combination, the drug combination is packaged in dosages that match a chrono-dosing regimen to match an optimal dose for the time of day. For example, in exemplary alternative embodiments, the beta adrenergic receptor antagonist or a beta blocker or equivalent, or a propranolol or equivalent; the non-steroidal anti-inflammatory drug or NSAID or equivalent, or etodolac or equivalent; and/or the gemcitabine and/or nab-paclitaxel, are packaged in dosages that match a chrono-dosing regimen to match an optimal dose for the time of day.

In exemplary alternative embodiments, the beta adrenergic receptor antagonist or beta blocker or equivalent or propranolol or equivalent; the non-steroidal anti-inflammatory drug or NSAID or equivalent or etodolac or equivalent; and/or the gemcitabine and/or nab-paclitaxel, are packaged in dosages that match a chrono-dosing regimen comprising:

(a) in the AM, 20 mg beta adrenergic receptor antagonist (a beta blocker), e.g., a propranolol or equivalent, 200mg NSAID, e.g., an etodolac or equivalent; in the afternoon, 10 mg beta blocker, 200 mg NSAID, e.g., an etodolac or equivalent; in the PM, 10 mg beta blocker, 400 mg NSAID;

(b) in the AM 40 mg beta adrenergic receptor antagonist (a beta blocker), e.g., a propranolol or equivalent, 200 mg NSAID, e.g., an etodolac or equivalent; in the afternoon 20 mg beta blocker, 200 mg NSAID; in the evening, 20 mg propranolol, 400 mg NSAID;

(c) in the AM 80 mg beta adrenergic receptor antagonist (a beta blocker), e.g., a propranolol or equivalent, 200 mg NSAID; in the afternoon 40 mg beta blocker, 200 mg NSAID, in the evening 40 mg, NSAID; or

(d) a dose escalation comprising a regimen of (a) to (b) to (c).

In exemplary alternative embodiments, the beta adrenergic receptor antagonist or beta blocker or equivalent or propranolol or equivalent; the non-steroidal anti-inflammatory drug or NSAID or equivalent or etodolac or equivalent; and/or the gemcitabine and/or nab-paclitaxel, are packaged in dosages that match a chrono-dosing regimen comprising:

Start: AM, 20 mg propranolol, 200 mg etodolac; afternoon, 10 mg propranolol, 200 mg etodolac; PM 5 mg propranolol, 400 mg etodolac;

Dose Escalation 1: AM 40 mg propranolol, 200 mg etodolac; afternoon 20 mg propranolol, 200 mg etodolac; evening, 10 mg propranolol, 400 mg etodolac;

Dose escalation 2: AM 80 mg propranolol, 200 mg etodolac; afternoon 40 mg propranolol, 200 mg etodolac, evening 20 mg, etodolac.

In exemplary alternative embodiments, the therapeutic drug combination is formulated for administration once a day, b.i.d. or t.i.d, or weekly, or biweekly, or monthly.

In exemplary alternative embodiments, the beta adrenergic receptor antagonist (a beta blocker) or propranolol or equivalent; the non-steroidal anti-inflammatory drug or NSAID or etodolac or equivalent; and/or the gemcitabine and/or nab-paclitaxel, are formulated for administration once a day, b.i.d. or t.i.d, or weekly, or biweekly, or monthly.

### Packaging

In alternative embodiments, provided are therapeutic combinations, preparations, formulations and/or kits, comprising combinations of ingredients, as described herein. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, provided are blister packs, clamshells or trays comprising a composition (e.g., a (the multi-ingredient combination of drugs as provided herein) combination of active ingredients) as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack as provided herein comprises a molded PVC base, with raised areas (the “blisters”) to contain the tablets, pills, etc. comprising the combinations as provided herein, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one aspect, a blister packs also comprise a method of packaging where the compositions comprising combinations of ingredients as provided herein are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. as provided herein are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two components (e.g., is a multi-ingredient combination of drugs as provided herein): a thermoformed “blister” which houses the product (e.g., a pharmaceutical combination as provided herein), and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb Ill.) using regular heat seal tooling. This alternative aspect, using heat seal tooling, can seal common types of thermoformed packaging.

### Blister Packaging

In alternative embodiments, provided are therapeutic combinations, preparations, formulations and/or kits can be manufactured as “blister packages” or as a plurality of packettes, including as lidded blister packages, lidded blister or blister card or packets or packettes, or a shrink wrap.

In alternative embodiments, laminated aluminum foil blister packs are used, e.g., for the preparation of drugs designed to dissolve immediately in the mouth of a patient. This exemplary process comprises having the drug combinations as provided herein prepared as an aqueous solution(s) which are dispensed (e.g., by measured dose) into an aluminum (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminum (e.g., alufoil) lidding material. In one aspect, hermetically-sealed high barrier aluminum (e.g., alufoil) laminates are used. In one aspect, any products of manufacture as provided herein, including kits or blister packs, use foil laminations and strip packs, stick packs, sachets and pouches, peelable and non-peelable laminations combining foil, paper, and film for high barrier packaging.

In alternative embodiments, any products of manufacture as provided herein, including kits or blister packs, include memory aids to help remind patients when and how to take the drug. This safeguards the drug's efficacy by protecting each pill until it's taken; gives the product or kit portability, makes it easy to take a dose anytime or anywhere. In alternative embodiments, each subcompartment is color coded, or coded for digital recognition, e.g., braille coded, or all compartments to be taken at the same time are coded in the same format (e.g., color).

The invention will be further described with reference to the following examples; however, it is to be understood that the invention is not limited to such examples.

### EXAMPLES

### Example 1

### Exemplary Treatment of Pancreatic Cancer by Administration of an Exemplary Combination Therapy

In alternative embodiments, provided are therapeutic combinations, pharmaceutical compositions, formulations, kits and devices for treating, preventing or ameliorating a tumor or a cancer, and methods for treating, preventing or ameliorating a tumor or a cancer, wherein in one embodiment the cancer is a pancreatic cancer, or in particular, a metastatic adenocarcinoma of the pancreas (mPCa). This example describes data demonstrating the efficacy of an exemplary therapeutic combination as described herein.

This exemplary study was designed to evaluate the safety, tolerability and efficacy of exemplary therapeutic combinations of the invention comprising propranolol and etodolac, formulated as VT-122 for this study, and gemcitabine and nab-paclitaxel, in the treatment of patients with locally advanced and metastatic pancreatic cancer.

Materials and Methods:

Patients with metastatic adenocarcinoma of the pancreas (mPCa) were eligible for this randomized investigator-initiated trial. Patients were administered propranolol and etodolac (PE) daily for one week prior to starting administration of gemcitabine and nab-paclitaxel (“GemNab”). Propranolol and etodolac (PE) was administered in a chronodosed regimen to maximize the therapeutic benefit and minimize side effects. The primary endpoint was survival.

Patient Selection:

Patients with treatment-naïve, locally advanced or metastatic pancreatic cancer were chosen; the median patient age was 62.8 years, and 68.2% male. The Eastern Cooperative Oncology Group (ECOG) prognostic score was between 0 to 2. Co-morbidities were required to be controlled prior to enrollment. All patients were required to be able to take tablets by mouth.

Study Design and Assessments:

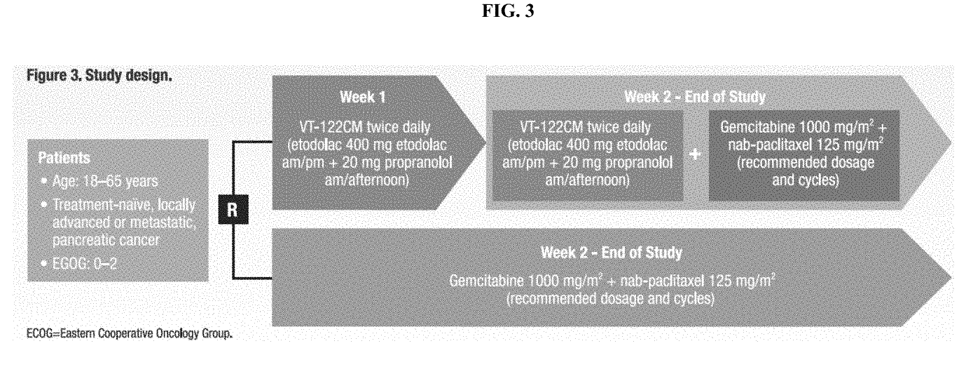

This was a single-center, randomized, open label study. As schematically illustrated in FIG. 3: twenty-three patients were randomized to receive either: (1) a placebo in addition to the gemcitabine and nab-paclitaxel (“GemNab”) combination (“the GemNab arm”); or, (2) administered the exemplary therapeutic combination of: gemcitabine and nab-paclitaxel (“GemNab”) and chrono-dosed propranolol and etodolac (PE) (“PEGenNab”) after one week of propranolol and etodolac (PE) alone (“the PEGemNab arm”). Pain at the time of diagnosis as reported in 80% of the patients in the (1) GemNab arm, and in 76.9% of the (2) PEGemNab arm, and jaundice was observed in 40% and 23% of the GemNab and PEGemNab arms, respectively.

The propranolol and etodolac (PE) were administered in the form of VT-122 (Vicus Therapeutics, Morristown N.J.); and administration was chronodosed at: etodolac twice daily, morning (AM) and evening (PM) at 400 mg per dose; and, 20 mg propranolol in the AM, and another 20 mg propranolol in the afternoon.

The gemcitabine and nab-paclitaxel (“GemNab”) were administered using standard dosage and administration, i.e., dosaging and administration regimen as on the FDA approved label (the FDA has approved nab-paclitaxel (ABRAXANE™) plus gemcitabine as a first-line treatment for patients with metastatic adenocarcinoma of the pancreas).

As illustrated in FIG. 3, the propranolol and etodolac (PE) were given alone the first week, and with gemcitabine and nab-paclitaxel (“GemNab”) in the second week, with gemcitabine at 1000 mg/m² and nab-paclitaxel at 125 mg/m².

Study Endpoints:

Primary endpoint=overall survival (OS), and selected secondary endpoints were progression-free survival (PFS), neuropathy, pain, body weight, and safety.

Results:

Summary:

Progression free survival was 7.2 and 11.8 months in the (1) GemNab arm and the (2) PEGemNab arm, respectively. Overall survival was 10.5 months for the (1) GemNab arm, and 15.9 months for the (2) PEGemNab arm. The treatment was well-tolerated with no unexpected adverse events.

Table 1, below, summarizes baseline disease characteristics:

TABLE 1
Baseline Disease Characteristics

| | VT-122CM + | |
|---|---|---|
| | Gemcitabine + | Gemcitabine + |
| | nab-paclitaxel | nab-paclitaxel |
| Characteristics | (n = 20) | (n = 17) |
| Male % | 70 | 59 |
| Mean age, y | 61.7 | 60.0 |
| Disease Stage, % | | |
| Locally advanced | 35 | 24 |
| Metastatic | 65 | 76 |
| Mean ECOG Score | 1.9 | 2.1 |
| Symptoms present, % | | |
| Pain | 90 | 88 |
| Weight loss | 65 | 47 |
| Jaundice | 30 | 65 |
| Vomiting | 33 | 24 |

^{a}Difference between groups, P = 0.03.
ECOG = Eastern Cooperative Oncology Group.

Survival:

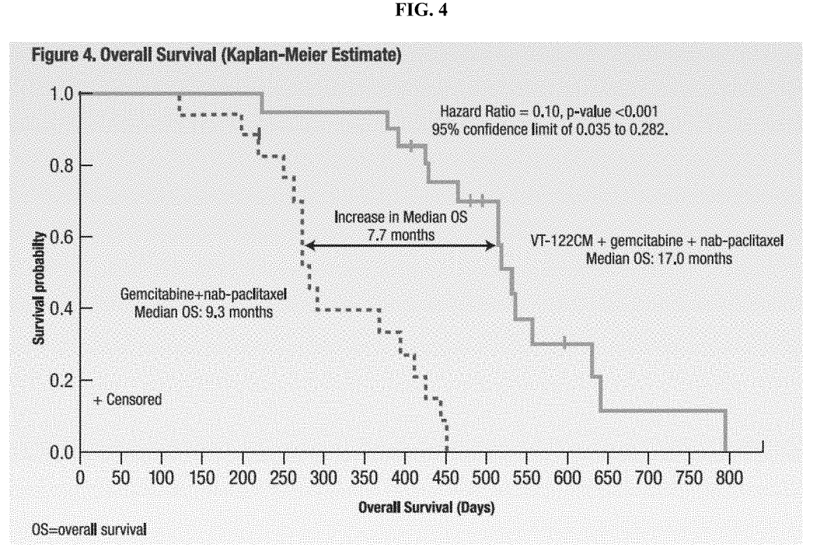

Overall survival in the VT-122-treated arm (2) was 17 months, which was significantly greater than the 9.3 months of the control arm (1); hazard ratio: 0.10 95% C1, 0.035% to 0.282; P<0.001. As graphically illustrated in FIG. 4, showing a Kaplan-Meier estimate, survival was increased by a significant 7.7 months.

Duration of Therapy:

Mean duration of chemotherapy on GemNab was 5.6 months longer in patients who also received the exemplary therapeutic combination also comprising VT-122 chronodosed, than in patients in the control group (no VT-122): 13.6 months versus 8.0 months, P<0.001.

Conclusions:

As compared to the (1) GemNab arm, administration of the (2) PEGemNab arm increased progression-free and overall survival. No unexpected side effected were seen.

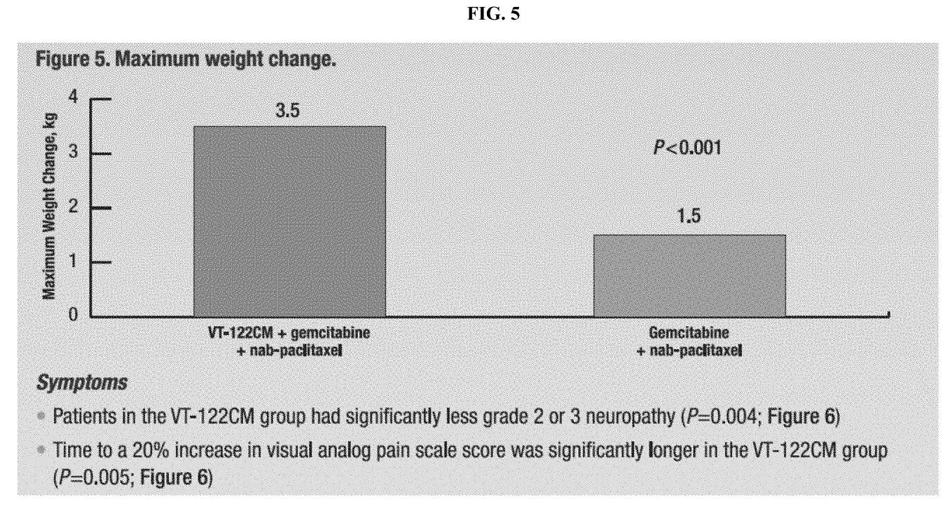

Also, as graphically illustrated in FIG. 5, the maximum weight gain was significantly greater in the VT-122 chronodosed group (arm 2).

Symptoms:

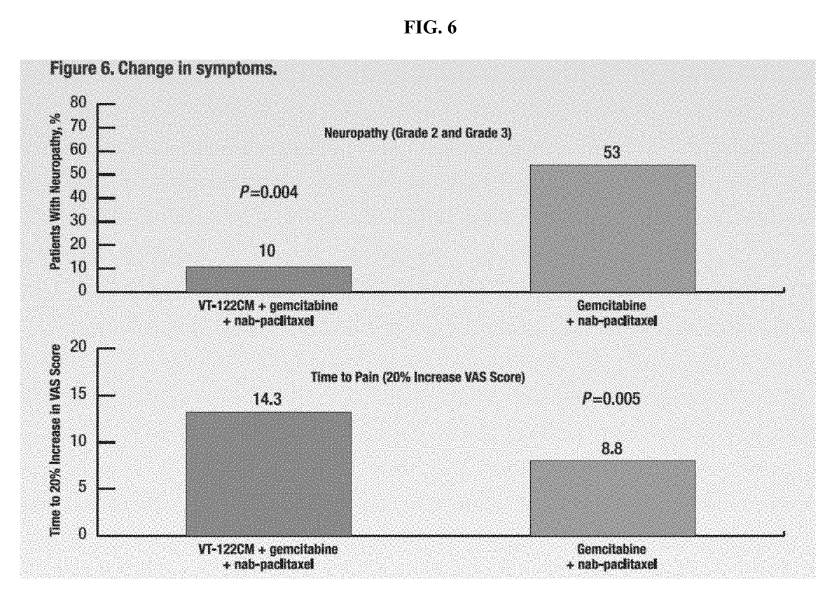

As graphically illustrated in FIG. 6, patients in the VT-122 chronodosed group (arm 2) had significantly less grade 2 or 3 neuropathy, P=0.004. Also as graphically illustrated in FIG. 6, time to a 20% increase in visual analog pain scale score was significantly longer in the VT-122 (arm 2) group, P=0.005.

In summary, the exemplary therapeutic combination comprising VT-122 chronodosed with gemcitabine and nab-paclitaxel was demonstrated to be effective in increasing overall survival (OS) by 7.7 months, nearly doubling the OS observed in a GemNab trial, and was demonstrated to be effective in increasing the duration of GemNab therapy in patients with locally advanced and metastatic pancreatic cancer. Additionally, use of propranolol and etodolac (PE) as a chronodosed VT-122 in this exemplary therapeutic combination reduced pain and neuropathy, and resulted in a greater increase in weight. The exemplary therapeutic combination comprising VT-122 did not result in serious adverse events.

A number of aspects of the invention have been described. Nevertheless, it will be understood that various modifications may be made without departing from the spirit and scope of the invention. Accordingly, other aspects are within the scope of the following claims.

What is claimed is:

1. A therapeutic combination of therapeutic agents or drugs for an individual in need thereof comprising or consisting of:
(a)
(i) a beta adrenergic receptor antagonist (a “beta blocker”);
(ii) a non-steroidal anti-inflammatory drug (a NSAID);
(iii) a gemcitabine or GEMZAR™; and,
(iv) a taxane, a paclitaxel, TAXOL™, ONXOL™, an albumin-bound paclitaxel (nab-paclitaxel) or ABRAXANE™, or any equivalent thereof;
(b) the therapeutic combination of therapeutic agents or drugs of (a), wherein the non-steroidal anti-inflammatory drug (a NSAID) comprises:
(i) a cyclooxygenase (COX) (or a prostaglandin synthase) inhibitor; or,
(ii) the COX inhibitor of (a), wherein the COX inhibitor comprises or consists of an etodolac or equivalent; a naproxen or equivalent; a celecoxib or equivalent; a rofecoxib or equivalent; a etoricoxib or equivalent; a valdecoxib or equivalent; a parecoxib or equivalent; a nabumetone or equivalent; a diclofenac (2-(2,6-dichloranilino) phenylacetic acid) or equivalent; or, a lumiracoxib or equivalent;
(c) the therapeutic combination of therapeutic agents or drugs of (a), wherein the beta adrenergic receptor antagonist (a beta blocker) comprises propranolol or equivalent, and optionally the propranolol is INDERAL™, AVLOCARDYL™, DERALIN™ DOCITON™, INDERALICI™, INNOPRAN XL™, or SUMIAL™; or
(d) the therapeutic combination of therapeutic agents or drugs of (a), wherein the therapeutic combination of an NSAID and beta blocker comprises or is a VT-122™ (Vicus Therapeutics, Morristown, N.J.).

2. The therapeutic combination of therapeutic agents or drugs of claim 1, wherein the etodolac is a LODINE™, LODINE SR™ or ECCOXOLAC™; or the celecoxib is CELEBREX™ or CELEBRA™; or the rofecoxib is VIOXX™, CEOXX™ or CEEOXX™; or the etoricoxib is ARCOXIA™, ALGIX™ or TAUXIB™; or the valdecoxib is BEXTRA™; the parecoxib is DYNASTAT™; the naproxen is XENOBID™, ALEVE™, ANAPROX™, MIRANAX™, NAPROGESIC™, NAPROSYN™, NAPRELAN™, PROXEN™ or SYNFLEX™; the nabumetone is RELAFEN™, RELIFEX™ or a GAMBARAN™; or, the diclofenac is FLECTOR PATCH™, VOLTAREN™, VOLTAROL™, DICLON™, DICLOFLEX DIFEN™, DIFENE™, CATAFLAM™, PENNSAID™, PANAMOR™, RHUMALGAN™, MODIFENAC™, ABITREN™, OLFEN™, VOVERAN™, ARTHROTEC™, DEDOLOR™, DEFLAMAT™, VETAGESIC™ or ZOLTEROL™.

3. The therapeutic combination of therapeutic agents or drugs of claim 1, further comprising an anti-cancer or anti-tumor antibody,
wherein optionally the anti-cancer or anti-tumor antibody is an alemtuzumab, a brentuximab vedotin, a cetuximab, a gemtuzumab ozogamicin, an abritumomab tiuxetan, a nimotuzumab, an ofatumumab, a panitumumab, a rituximab, a tositumomab, or a trastuzumab.

4. The therapeutic combination of therapeutic agents or drugs of claim 1, further comprising a cytokine,
wherein optionally the cytokine comprises an IL-2 or an interferon (IFN),
and optionally the interferon is an alpha-IFN or a gamma-IFN;
and optionally the IL-2 is a recombinant IL-2, an aldesleukin, or a PROLEUKIN (Prometheus Laboratories),
wherein optionally the IL-2, recombinant IL-2, or aldesleukin is dosages at about: 1 to 20, 2 to 10, 4 to 5, or 4.5 millions of IUs per cycle; or is dosaged for: 1 to 5, 2 to 4, or 3 cycles number of cycles of therapy.

5. The therapeutic combination of therapeutic agents or drugs of claim 1, further comprising a chemotherapeutic agent,
wherein optionally the chemotherapeutic agent comprises a doxorubicin or a carboplatin, or comprises an inducer of apoptosis or a mitotic inhibitor or anti-microtubule inhibitor, or an alkylating agent, or a topoisomerase inhibitor, or a glycopeptide antibiotic, or steroid receptor inhibitor, or a matrix metalloproteinase (MMP) inhibitor, or an mTOR (mammalian target of rapamycin) inhibitor, or a macrolide or a composition comprising a macrolide ring,
and optionally the inducer of apoptosis or a mitotic inhibitor or anti-microtubule inhibitor comprises or consists of a raltitrexed or equivalent, or TOMUDEX™; a doxorubicin or equivalent, or ADRIAMYCIN™; a fluorouracil or 5-fluorouracil or equivalent; a docetaxel or equivalent, or TAXOTERE™; a larotaxel, tesetaxel or ortataxel or equivalent; an epothilone or an epothilone A, B, C, D, E or F or equivalent; an ixabepilone (also known as azaepothilone B) or equivalent, or BMS-247550™; a vincristine (also known as leurocristine) or equivalent, or ONCOVIN™; a vinblastin, vinblastine, vindesine, vinflunine, vinorelbine or NAVELBINE™ or equivalent; or, any combination thereof,
and optionally the alkylating agent comprises or consists of a cisplatin or equivalent; a cisplatinum or equivalent; a cis-diamminedichloridoplatinum(II) (CDDP) or equivalent; a carboplatin or equivalent; a oxaloplatin or equivalent; a cyclophosphamide (cytophosphane) or equivalent, or ENDOXAN™, CYTOXAN™, NEOSAR™ or REVIMMUNE™; a mechlorethamine or equivalent; a chlormethine or equivalent; a mustine or equivalent; a nitrogen mustard or equivalent; a chlorambucil or equivalent, or LEUKERAN™; or, a combination thereof,
and optionally the topoisomerase inhibitor comprises or consists of an etoposide or equivalent, or EPOSIN™, ETOPOPHOS™, VEPESID™ or VP-16™; an amsacrine or equivalent; a topotecan or equivalent, or HYCAMTIN™; a teniposide or equivalent, or VUMON™ or VM-26™; an epipodophyllotoxin or equivalent; a camptothecin or equivalent; an irinotecan or equivalent, or CAMPTOSAR™; or, a combination thereof, and optionally the glycopeptide antibiotic comprises or consists of a bleomycin or equivalent or a bleomycin A2 or B2 or equivalent; a mitomycin or a mitomycin C or equivalent, a plicamycin (also known as mithramycin) or equivalent, or MITHRACIN™; or, a combination thereof,
and optionally the steroid receptor inhibitor comprises or consists of an estrogen receptor modulator (a SERM), and optionally the estrogen receptor modulator comprises or consists of a tamoxifen or equivalent, or NOLVADEX™, ISTUBAL™ or VALODEX™, and optionally the steroid inhibitor or an anti-steroid comprises or consists of a finasteride or equivalent, or PROSCAR™, PROPECIA™, FINCAR™, FINPECIA™, FINAX™, FINAST™, FINARA™, FINALO™, PROSTERIDE™, GEFINA™, APPECIA™, FINASTERID IVAX™, FINASTERID or ALTERNOVA™,
and optionally the macrolide or composition comprising a macrolide ring comprises or consists of a clarithromycin or equivalent, or BIAXIN™, KLARICID™, KLABAX™, CLARIPEN™, CLARIDAR™, FROMILID™ or CLACID™; an azithromycin or equivalent, or ZITHROMAX™, ZITROMAX™ or SUMAMED™; a dirithromycin or equivalent; an erythromycin or equivalent; a roxithromycin or equivalent, or ROXO™, SURLID™, RULIDE™, BIAXSIG™, ROXAR™, ROXIMYCIN™ or COROXIN™; a telithromycin or equivalent or KETEK™; a josamycin or equivalent; a kitasamycin or equivalent; a midecamycin or equivalent; oleandomycin or equivalent; a roxithromycin or equivalent, or ROXO™, SURLID™, RULIDE™, BIAXSIG™, ROXAR™, ROXIMYCIN™ or COROXIN™; a troleandomycin or equivalent; or a tylosin or equivalent; or, any combination thereof,
wherein optionally the chemotherapeutic agent comprises a sorafenib or equivalent, or NEXAVAR™; a sunitinib or equivalent, or SUTENT™; an erlotinib or equivalent, or TARCEVA™; an imatinib or equivalent, or GLEEVEC™; a lapatinib or equivalent, or TYKERB™; a toceranib or equivalent, or PALLADIA™; a masitinib or equivalent, or MASIVET™, a bevacizumab or equivalent, or AVASTIN™; a trastuzumab or equivalent, or HERCEPTIN™; a cetuximab or equivalent, or ERBITUX™; a bevacizumab or equivalent, or AVASTIN™ or BMW 2992; a gefitinib or equivalent, or IRESSA™; a ranibizumab or equivalent, or LUCENTIS™; a pegaptanib or equivalent, or MACUGEN™; a dasatinib or equivalent, or BMS-354825™; a sunitinib or equivalent, or SUTENT™; a pazopanib or equivalent; a nilotinib or equivalent, or TASIGNA™; a panitumumab or equivalent, or VECTIBIX™; a bandetinib or equivalent; a brivanib or equivalent, or E7080™; a thalidomide or equivalent, or THALOMID™; lenalidomide or equivalent, or REVLIMID™; a bortezomib or equivalent, or VELCADE™; disulfiram or equivalent, or ANTABUSE™ or ANTABUS™; or an epigallocatechin gallate (EGCG) or equivalent; a demecolcine, an etoglucid or elsamitrucin, a lonidamine, a lucanthone, a mitotane or a mitoguazone or equivalent; or any combination thereof.

6. The therapeutic combination of therapeutic agents or drugs of claim 1, further comprising a radiotherapy enhancing agent.

7. The therapeutic combination of therapeutic agents or drugs of claim 1, further comprising a proton pump inhibitor (a PPI),
wherein optionally the proton pump inhibitor comprises or consists of a benzimidazole compound or structure, or an imidazopyridine compound or structure.

8. The therapeutic combination of therapeutic agents or drugs of claim 1, further comprising a radioactive particle or isotope; or a microscopic, radioactive glass microsphere, or a TheraSphere; or a drug-eluting or a cancer drug-eluting particle, liposome or bead, or a doxorubicin-loaded drug-eluting bead, or a DC Bead®.

9. The therapeutic combination of therapeutic agents or drugs of claim 1, further comprising an adjuvant.

10. The therapeutic combination of therapeutic agents or drugs of claim 1, wherein two or more drugs of the therapeutic combination are formulated as separate compositions, or two or more drugs of the therapeutic combination are formulated into one composition or drug formulation (two or more drugs of the therapeutic combination are formulated together).

11. The therapeutic combination of therapeutic agents or drugs of claim 10, wherein the beta adrenergic receptor antagonist, or the beta blocker or equivalent, or the propranolol or equivalent; the non-steroidal anti-inflammatory drug, or the NSAID or equivalent, or the etodolac or equivalent; the gemcitabine or GEMZAR™; and the taxane, the paclitaxel, TAXOL™, ONXOL™, the albumin-bound paclitaxel (nab-paclitaxel) or ABRAXANE™ or any equivalent thereof, are all formulated or packaged in different compositions or formulations, in a single formulation or package, or in paired combinations or packages,
and optionally are all administered separately or are all administered together,
and optionally the beta adrenergic receptor antagonist, the beta blocker or equivalent, or the propranolol or equivalent and the NSAID or equivalent, or the etodolac or equivalent, are administered or packaged together either in separate formulations or packages or a single formulation or package,
and optionally the gemcitabine or GEMZAR™; and the taxane, the paclitaxel, TAXOL™, ONXOL™, the albumin-bound paclitaxel (nab-paclitaxel) or ABRAXANE™ or any equivalent thereof, are administered or packaged together either in separate formulations or packages or a single formulation or packages;
(b) the beta adrenergic receptor antagonist, or a beta blocker or equivalent, or a propranolol or equivalent; and the non-steroidal anti-inflammatory drug, or a NSAID or equivalent, or an etodolac or equivalent are formulated in different compositions or formulations, or, are formulated in the same composition or formulation, or are formulated together.

12. The therapeutic combination of therapeutic agents or drugs of claim 1, wherein
(a) one or two or more or all of the drugs of the therapeutic combination are packaged individually, or are packaged together, or packaged in any combination, in a single package, a plurality of packages or packettes, or packaged as a blister packet, lidded blister or blister card or packets, or a shrink wrap,
and optionally the beta adrenergic receptor antagonist, or a beta blocker or equivalent, or a propranolol or equivalent; the non-steroidal anti-inflammatory drug, or a NSAID or equivalent, or an etodolac or equivalent; and the therapeutic agent for the treatment of cancer, are packaged individually in a single package, a plurality of packages or packettes, or a blister packet, lidded blister or blister card or packets, or a shrink wrap;
(b) one or two or more or all of the drugs of the therapeutic combination are packaged together or in any combination in a single package, a plurality of packages or packettes, or a blister packet, lidded blister or blister card or packets, or a shrink wrap,
and optionally two, three or more or all of the drugs are released upon opening of the single package, plurality of packages or packettes, blister packet, lidded blister, blister card or packets or shrink wrap; or
(c) the beta adrenergic receptor antagonist, or a beta blocker or equivalent, or a propranolol or equivalent; the non-steroidal anti-inflammatory drug, or a NSAID or equivalent, or an etodolac or equivalent; and the therapeutic agent for the treatment of cancer are packaged together in a single package, a plurality of packages or packettes, or a blister packet, lidded blister or blister card or packets, or a shrink wrap, and two or more or all of the drugs are released upon opening of the single package, plurality of packages or packettes, blister packet, lidded blister, blister card or packets or shrink wrap.

13. The therapeutic combination of therapeutic agents or drugs of claim 1, wherein one or two or more or all of the drugs of the therapeutic combination are formulated or manufactured as a parenteral formulation, an aqueous solution, a liposome, an injectable solution, a tablet, a pill, a lozenge, a capsule, a caplet, a patch, a spray, an inhalant, a powder, a freeze-dried powder, an inhalant, a patch, a gel, a geltab, a nanosuspension, a nanoparticle, a nanoliposome, a microgel, a pellet, a suppository or any combination thereof,
and optionally the therapeutic combination is formulated or manufactured together in one parenteral formulation, one aqueous solution, one liposome, one injectable solution, one freeze-dried powder, one feed, one food, one food supplement, one pellet, one lozenge, one liquid, one elixir, one aerosol, one inhalant, one adhesive, one spray, one powder, one freeze-dried powder, one patch, one tablet, one pill, one capsule, one gel, one geltab, one lozenge, one caplet, one nanosuspension, one nanoparticle, one nanoliposome, one microgel or one suppository.

14. The therapeutic combination of therapeutic agents or drugs of claim 1, wherein
(a) the dosage of etodolac ranges from about 200 mg to 400 mg a day, or, about 10, 15, 20, 25, 30, 35, 40, 45, 50, 75, 80, 85, 90, 100, 150, 200, 250, 300, 350, 400, 450, 500, 600, 700, 800, 900 or 1000 mg or more;
(b) the dosage of propranolol ranges from 10 to 320 mg per day based on heart rate and blood pressure of the individual, or, about 10, 15, 20, 25, 30, 35, 40, 45, 50, 75, 80, 85, 90, 100, 150, 200, 250, 300, 350, 400, 450, 500, 600, 700, 800, 900 or 1000 mg or more;
(c) the drug combination is packaged in dosages that match a chrono-dosing regimen to match an optimal dose for the time of day;
(d) the beta adrenergic receptor antagonist or a beta blocker or equivalent, or a propranolol or equivalent; the non-steroidal anti-inflammatory drug or NSAID or equivalent, or etodolac or equivalent; and also optionally the gemcitabine or GEMZAR™ and/or the taxane, the paclitaxel, TAXOL™, ONXOL™, the albumin-bound paclitaxel (nab-paclitaxel) or ABRAXANE™, or any equivalent thereof, are packaged in dosages that match a chrono-dosing regimen to match an optimal dose for the time of day;
(e) the beta adrenergic receptor antagonist or beta blocker or equivalent or propranolol or equivalent; the non-steroidal anti-inflammatory drug or NSAID or equivalent or etodolac or equivalent; and also optionally the gemcitabine or GEMZAR™ and/or the taxane, the paclitaxel, TAXOL™, ONXOL™, the albumin-bound paclitaxel (nab-paclitaxel) or ABRAXANE™, or any equivalent thereof, are packaged in dosages that match a chrono-dosing regimen comprising:
(i) in the AM, 20 mg beta adrenergic receptor antagonist (a beta blocker), e.g., a propranolol or equivalent, 200mg NSAID, e.g., an etodolac or equivalent; in the afternoon, 10 mg beta blocker, 200 mg NSAID, e.g., an etodolac or equivalent; in the PM, 10 mg beta blocker, 400 mg NSAID;
(ii) in the AM 40 mg beta adrenergic receptor antagonist (a beta blocker), e.g., a propranolol or equivalent, 200 mg NSAID, e.g., an etodolac or equivalent; in the afternoon 20 mg beta blocker, 200 mg NSAID; in the evening, 20 mg propranolol, 400 mg NSAID;
(iii) in the AM 80 mg beta adrenergic receptor antagonist (a beta blocker), e.g., a propranolol or equivalent, 200 mg NSAID; in the afternoon 40 mg beta blocker, 200 mg NSAID, in the evening 40 mg, NSAID; or
(iv) a dose escalation comprising a regimen of (a) to (b) to (c),
and optionally the beta adrenergic receptor antagonist or beta blocker or equivalent or propranolol or equivalent; the non-steroidal anti-inflammatory drug or NSAID or equivalent or etodolac or equivalent; and also optionally the gemcitabine or GEMZAR™ and/or the taxane, the paclitaxel, TAXOL™, ONXOL™, the albumin-bound paclitaxel (nab-paclitaxel) or ABRAXANE™, or any equivalent thereof, are packaged in dosages that match a chrono-dosing regimen comprising:
Start: AM, 20 mg propranolol, 200mg etodolac; afternoon, 10 mg propranolol, 200 mg etodolac; PM 5 mg propranolol, 400 mg etodolac;
Dose Escalation 1: AM 40 mg propranolol, 200 mg etodolac; afternoon 20 mg propranolol, 200 mg etodolac; evening, 10 mg propranolol, 400 mg etodolac;
Dose escalation 2: AM 80 mg propranolol, 200 mg etodolac; afternoon 40 mg propranolol, 200 mg etodolac, evening 20 mg, etodolac;
(f) the therapeutic drug combination is formulated for administration once a day, b.i.d. or t.i.d, or weekly, or biweekly, or monthly; or
(g) the beta adrenergic receptor antagonist (a beta blocker) or propranolol or equivalent; the non-steroidal anti-inflammatory drug or NSAID or etodolac or equivalent; and also optionally the gemcitabine or GEMZAR™ and/or the taxane, the paclitaxel, TAXOL™, ONXOL™, the albumin-bound paclitaxel (nab-paclitaxel) or ABRAXANE™ or any equivalent thereof, are formulated for administration once a day, b.i.d. or t.i.d, or weekly, or biweekly, or monthly.

15. The therapeutic combination of therapeutic agents or drugs of claim 1, wherein the therapeutic combination of drugs are formulated for administration intravenously, topically, orally, by inhalation, by infusion, by injection, by inhalation, intraperitoneally, intramuscularly, subcutaneously, intra-aurally, for intra-articular administration, for intra-mammary administration, for topical administration or for absorption through epithelial or mucocutaneous linings.

16. A device, a medical device, an implant, a breast implant, a prosthesis, a stent, a catheter, comprising a therapeutic combination of therapeutic agents or drugs of claim 1.

17. A pharmaceutical composition or formulation comprising the therapeutic combination of therapeutic agents or drugs of claim 1,
and optionally the pharmaceutical composition or formulation further comprises a pharmaceutically acceptable excipient, wherein optionally the pharmaceutical composition or formulation is formulated or manufactured as a feed, a food, a food or feed concentrate, a pellet, a lozenge, a liquid, a lotion, an implant, a nanoparticle, an elixir, an aerosol, a spray, an aerosol, an inhalant, a powder, a tablet, a pill, a capsule, a gel, a geltab, a nanosuspension, a nanoparticle, a patch, a microgel or a suppository.

18. A method for treating, preventing or ameliorating a tumor or a cancer, comprising: applying or administering to an individual in need thereof; or, applying or administering to an effected tissue: the therapeutic combination of therapeutic agents or drugs of claim 1,
wherein optionally the therapeutic agents or drugs are administered separately or together, or at the same time, or in synchrony, or by chrono-dosing, or one of the therapeutic agents or drugs is administered before another of the therapeutic agents or drugs,
and optionally the therapeutic agents or drugs are formulated for administration intravenously (IV), parenterally, nasally, topically or locally, orally, or by liposome, implant or vessel-targeted nanoparticle delivery,
and optionally the cancer or tumor is: a pancreatic cancer, an adenocarcinoma, a mastocytoma or a mast cell tumor, an ovarian cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm' s tumor, ovarian carcinoma, a bone cancer, an osteosarcoma, a renal cancer, or head and neck cancer, oral cancer, a laryngeal cancer, or an oropharyngeal cancer,
optionally further comprising: an anti-cancer or anti-tumor radiotherapy or a proton beam therapy.

19. A method for treating, preventing or ameliorating a tumor or a cancer, comprising:
(a) applying or administering to an individual in need thereof; or, applying or administering to an effected tissue; the therapeutic combination of therapeutic agents or drugs of claim 1,
wherein optionally the therapeutic agents or drugs are administered separately or together, or at the same time, or in synchrony, or by chrono-dosing, or one of the therapeutic agents or drugs is administered before another of the therapeutic agents or drugs,
and optionally the therapeutic agents or drugs are formulated for administration intravenously (IV), parenterally, nasally, topically or locally, orally, or by liposome, implant or vessel-targeted nanoparticle delivery; and
(b) administering to the individual in need thereof:
(i) a systemic anti-cancer or anti-tumor treatment, wherein optionally the systemic anti-cancer or anti-tumor treatment comprises administration of a drug, a biologic, a nutrient, an anti-cancer or anti-tumor dietary regimen, a radioactive agent, a tumor ablative agent, or
(ii) an anti-cancer or anti-tumor radiotherapy or a proton beam therapy,
wherein the therapeutic combination or pharmaceutical composition or formulation of (a) is administered before the anti-cancer or anti-tumor treatment of (b), or both are administered consecutively, or the therapeutic combination or pharmaceutical composition or formulation of (a) is administered after the anti-cancer or anti-tumor treatment of (b), or any combination thereof,
optionally further comprising: an anti-cancer or anti-tumor radiotherapy or a proton beam therapy.
